(19) United States
(12) Patent Application Publication
(10) Pub. No.: US 2019/0038446 A1
(43) Pub. Date: Feb. 7, 2019

(54) HEMORRHOID KIT

(71) Applicant: Nicole Takemoto, Ewa Beach, HI (US)

(72) Inventor: Nicole Takemoto, Ewa Beach, HI (US)

(21) Appl. No.: 16/056,477

(22) Filed: Aug. 6, 2018

Related U.S. Application Data
(60) Provisional application No. 62/541,254, filed on Aug. 4, 2017.

(51) Int. Cl. A61F 5/00; A61F 5/34; A61F 7/08; A61F 7/10; A41B 9/00

(52) U.S. Cl. CPC A61F 5/0093; A61F 5/34; A61F 2007/0028; A61F 7/10; A41B 9/001; A61F 7/08

(57) ABSTRACT

A kit for providing pain relief to a user suffering from rectal, anal, colon discomforts, infections or diseases is disclosed herein. The kit for providing pain relief to a user suffering from rectal, anal, colon discomforts, infections or diseases includes at least one cushion-device, at least one cold therapy-device and one or more medicaments. In a preferred embodiment, the kit includes cushioned seats, rectal plugs, underwear with pockets for holding ice-packs therein, lotions, and suppositories.

### CROSS REFERENCE TO RELATED APPLICATION

The present application is related to and claims priority to U.S. Provisional Patent Application No. 62/541,254 filed Aug. 4, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. FIELD OF THE INVENTION

The present invention relates generally to the field of rectal, anal, colon discomforts, infections or diseases and more specifically relates to a kit for treating and soothing rectal, anal, colon discomforts, infections or diseases, particularly hemorrhoids.

### 2. DESCRIPTION OF RELATED ART

Hemorrhoids, also called piles, are vascular structures in the anal canal. They become a disease when swollen or inflamed. The signs and symptoms of hemorrhoids depend on the type present. Internal hemorrhoids often result in painless, bright red rectal bleeding when defecating. External hemorrhoids often result in pain and swelling in the area of the anus. Hemorrhoids often cause chronic pain and suffering to individuals and it can be difficult for individuals to find relief. When looking for relief, they may find many available products, but have no way of knowing what to use and how. With so many options, it is overwhelming for individuals to try and research the right products and remedies. A suitable solution is desired.

U.S. Pat/Pub. No. 2007/0078417 to J. Stephen Bohan relates to a method and system for systematically treating hemorrhoids. The described method and system for systematically treating hemorrhoids includes educational materials about hemorrhoids including the prevention and treatment thereof; cream for use in early stages of development for relief of minor symptoms in a manner that aids in preventing progression to more uncomfortable stages requiring surgical intervention, fiber pad wipes, cleanser, and optionally, a prescription strength ointment for relief of moderately severe symptoms of pain and swelling, the use of which can prevent progression to a stage requiring surgery.

### BRIEF SUMMARY OF THE INVENTION

In view of the foregoing disadvantages inherent in the known hemorrhoids art, the present disclosure provides a novel hemorrhoid kit. The general purpose of the present disclosure, which will be described subsequently in greater detail, is to provide a kit including various objects configured to treat and soothe rectal, anal, colon discomforts, infections or diseases, particularly hemorrhoids.

A kit is disclosed herein. The kit includes at least one cushion-device configured to provide comfort to at least one body-part of the user, at least one cold-therapy device configured to soothe inflammation associated with the rectal, anal, colon discomforts, infections or diseases, and one or more medicaments configured for application to the at least one body-part of the user.

A method of using the kit is also disclosed herein. The method of using the kit may comprise the steps of: providing the kit as above and using one of the at least one cushion-device, the at least one cold-therapy device, and the at least one medicament to provide pain relief from the rectal, anal, colon discomforts, infections or diseases.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a hemorrhoid kit, constructed and operative according to the teachings of the present disclosure.

FIG. 1 is a front perspective view of the kit during an ‘in-use’ condition, according to an embodiment of the disclosure.

FIG. 2A is a side perspective view of the kit of FIG. 1, according to an embodiment of the present disclosure.

FIG. 2B is a bottom perspective view of the kit of FIG. 1, according to an embodiment of the present disclosure.

FIG. 3 is a top perspective view of the kit of FIG. 1, according to an embodiment of the present disclosure.

FIG. 4 is a side perspective view of the kit of FIG. 1, according to an embodiment of the present disclosure.

FIG. 5 is a side-front perspective view of the kit of FIG. 1, according to an embodiment of the present disclosure.

FIG. 6 is a flow diagram illustrating a method of use for kit, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to hemorrhoids and more particularly to a hemorrhoid kit as used to improve kits for treating and soothing hemorrhoids.

Generally, disclosed is a kit designed to help individuals find immediate pain relief from rectal, anal, colon discomforts, infections or diseases, particularly hemorrhoids. It offers a variety of remedies, as well as product knowledge and a user guide to assist individuals in obtaining relief. It may afford individuals different products, such as ice-packed underwear, an inflatable bathtub seat, and an anal plug to assist in reducing inflammation and discomfort, enabling users to find various hemorrhoid treatments in one convenient location. The kit may be used to heal and restore the affected area and aid in everyday activities while dealing with hemorrhoids.

The kit may include inflatable neck/back/buttocks rest with suction cups for use in the bathtub, an anal plug/applicator, a manual/guide, hemorrhoid cream and aloe, and underwear with a front and back pouch/pocket to insert ice packs. The inflatable seat may be used for soaking and relaxing the painful areas. The underwear may be reusable ad provides various areas with the cooling effects of ice. The plug may be put in the freezer and then used in the anus for relief. The hemorrhoid cream may be a hemp oil that helps with internal inflammation. The kit may include any of the following: cushions, donuts, seats, Epsom salts, diapers, underwear, rectum inserts, applicators, plugs, plants, oils, creams, lotions, suppositories, tools, medical prescriptions, kit manual guide, book, instructions, etc. The exact specifications may vary.

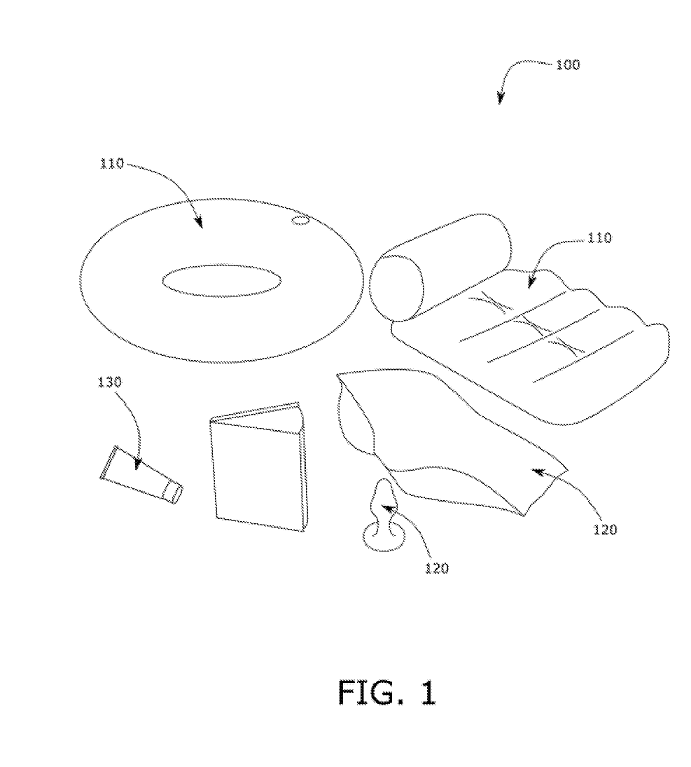

Referring now more specifically to the drawings by numerals of reference, there is shown in FIGS. 1-6, various views of a kit 100.

FIG. 1 shows a kit 100 according to an embodiment of the present disclosure. As Illustrated, the kit 100 may include at least one cushion-device 110, at least one cold-therapy device 120 and one or more medicaments 130. The kit 100 may be used for providing pain relief to a user suffering from rectal, anal, colon discomforts, infections or diseases, particularly hemorrhoids.

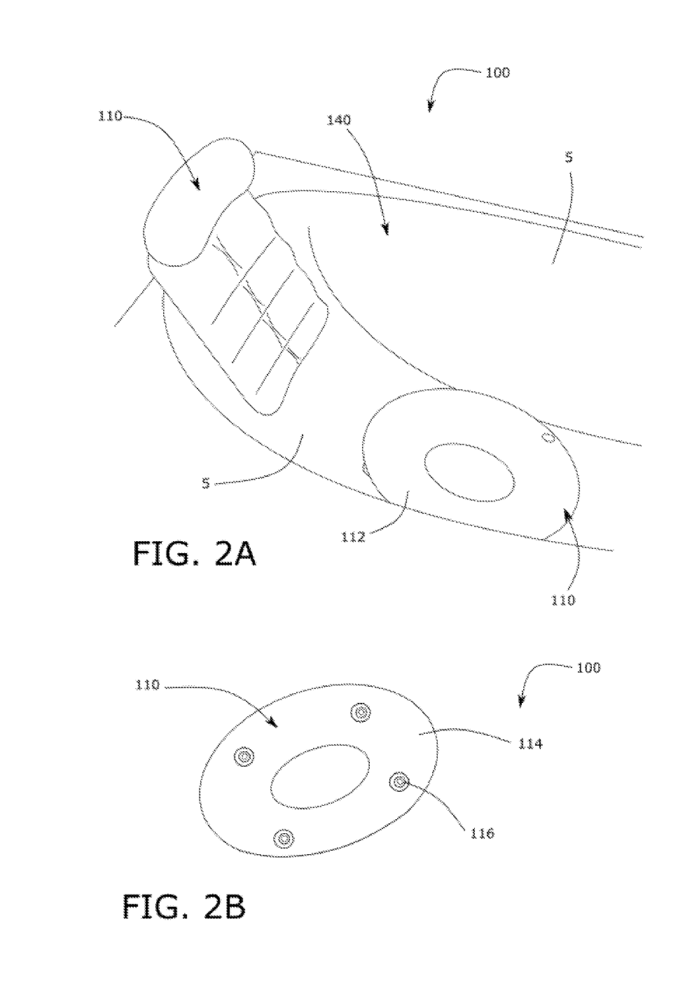

FIGS. 2A-2B show a side and bottom perspective views of the kit 100 of FIG. 1, according to an embodiment of the present disclosure. The at least one cushion-device 110 may be configured to provide comfort to at least one body-part of the user. In a preferred embodiment, the at least one cushion-device 110 may be inflatable. In this embodiment, the at least one cushion-device 110 may include an inner-volume configured to receive air, water, etc. In another example, the at least one cushion-device 110 may include an inner-volume having separate fillable chambers configured to receive the air, the water, etc. The inner-volume of the at least one cushion-device 110 may be defined by an outer-shell. In one embodiment, the outer-shell may include a soft material thereon to provide added comfort. The soft material may be waterproof.

Further, as shown the at least one cushion-device 110 may include a donut-shape (FIG. 2B). This may allow the user to target particular areas of their body to soothe. In another embodiment, the at least one cushion-device 110 may be circular, square, rectangle, etc. and may be shaped as a seat to allow the user to sit thereon and soothe the area. In addition to this, the cushion-device 110 may include a front-surface 112 and a rear-surface 114. An attachment-means 116 may be located on a rear-surface 114 of the cushion-device 110 and configured to attach the cushion-device 110 to a third surface 5. In the preferred embodiment, the third surface 5 is at least one of a wall of a bathtub and a bottom of a bathtub. Further, in the preferred embodiment, the attachment-means 116 may be at least one suction-cup. However, it should be appreciated that other attachment-means 116 may be utilized. The chosen attachment-means 116 may vary depending on the third surface 5.

In addition, in one embodiment, the kit 100 may further comprise at least one warm-therapy device 140. The at least one warm-therapy device 140 may be particularly useful for use with the at least one cushion-device 110, because the bathtub may serve as the at least one warm-therapy device 140. Soothing items such as Epsom salts may also be provided for use with the bathtub. In other embodiments, the at least one warm-therapy device 140 may be a smaller bath device sized for the user to sit therein. Other examples of the at least one warm-therapy device 140 may include heat lamps, heat lights, heating pads, and the like.

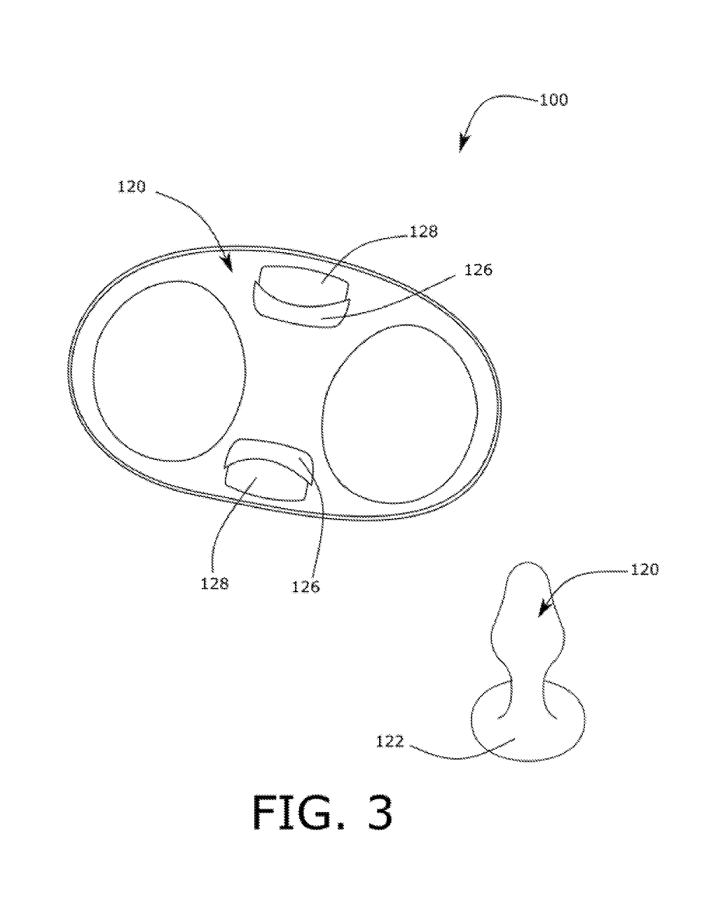

FIG. 3 shows a front perspective view of the kit 100 of FIG. 1, according to an embodiment of the present disclosure. The at least one cold-therapy device 120 may be configured to soothe inflammation associated with the hemorrhoids. In the preferred embodiment, the at least one cold-therapy device 120 may include a rectal plug 122. The rectal plug 122 may be made from an insulative-material configured to maintain a temperature. The user may store the rectal plug 122 in a freezer until the rectal plug 122 has reached a desired temperature, and then may insert the rectal plug 122 into the rectum to soothe the hemorrhoids. Variations of the rectal plugs 122 are also contemplated, such as rectal inserts, rectal applicators, etc.

As shown further in FIG. 3, the at least one cold-therapy device 120 may further include underwear 124 that has at least one pocket 126. The underwear 124 may be adult underwear 124, children's underwear 124 or diapers. In the preferred embodiment, the at least one pocket 126 may be two pockets 126: one located on a rear of the underwear 124, and one located on a front of the underwear 124. In one embodiment, the kit 100 may further include an ice-pack 128 configured for removable-insertion into the at least one pocket 126. In the preferred embodiment, the kit 100 may provide two ice-packs 128, one for insertion into the pocket 126 on the rear of the underwear 124 and one for insertion into the pocket 126 on the front of the underwear 124. The user may store the ice-packs 128 in the freezer and insert into the pockets 126 when needed to soothe the hemorrhoids.

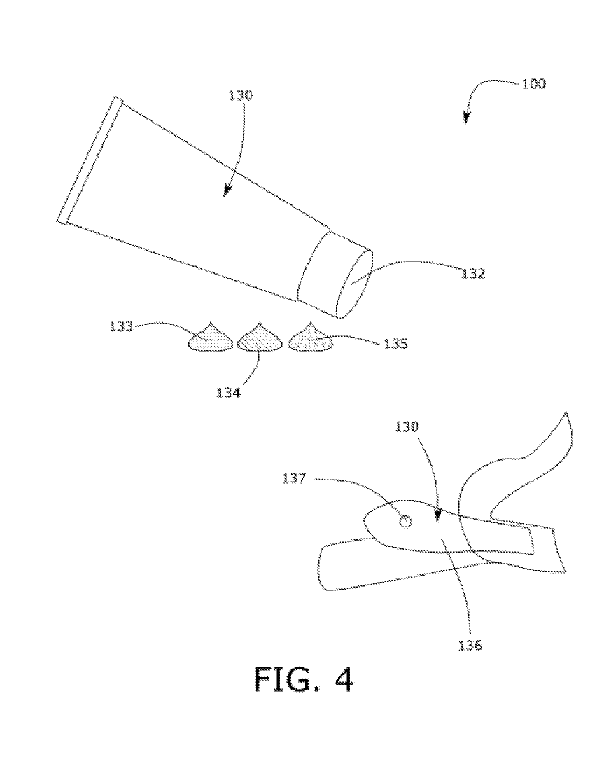

FIG. 4 shows a front perspective view of the kit 100 of FIG. 1, according to an embodiment of the present disclosure. The medicaments 130 may be configured for application to the hemorrhoids. In one embodiment, a medicament 130 of the one or more medicaments 130 may include at least one lotion 132. The at least one lotion 132 may include a variety of ingredients chosen for their soothing and anti-inflammatory properties to soothe the hemorrhoids. For example, in one embodiment, the at least one lotion 132 may include hemp oil 133, aloe vera 134, and anti-inflammatory medicine 135. In some embodiments, the at least one lotion 132 is contemplated to include any of the hemp oil 133, aloe vera 134 and anti-inflammatory medicine 135, a combination of two, or a combination of all three. Further ingredients are also contemplated to be added to the lotion 132. In other embodiments, prescription lotions may be used. In addition, other variants of lotions 132 are envisioned, such as creams, oils, balms, pomades, essences, etc.

Further, as shown, in another embodiment, a medicament 130 of the one or more medicaments 130 may include at least one suppository 136. The at least one suppository 136 may be inserted into the rectum for soothing and aiding in the betterment of the hemorrhoids. The at least one suppository 136 may include the ingredient phenylephrine 137. The phenylephrine 137 in particular may activate vasoconstriction and temporarily reduce swelling, burning, pain, and itching associated with the hemorrhoids. Other soothing ingredients may be included in the at least one suppository 136 such as cocoa butter, mineral oil, shark liver oil, corn starch, etc. It should, however, be appreciated that the at least one suppository 136 is not limited to any of the ingredients listed above, including phenylephrine 137, and that any future medicament 130 that is not currently available in medicine are also contemplated.

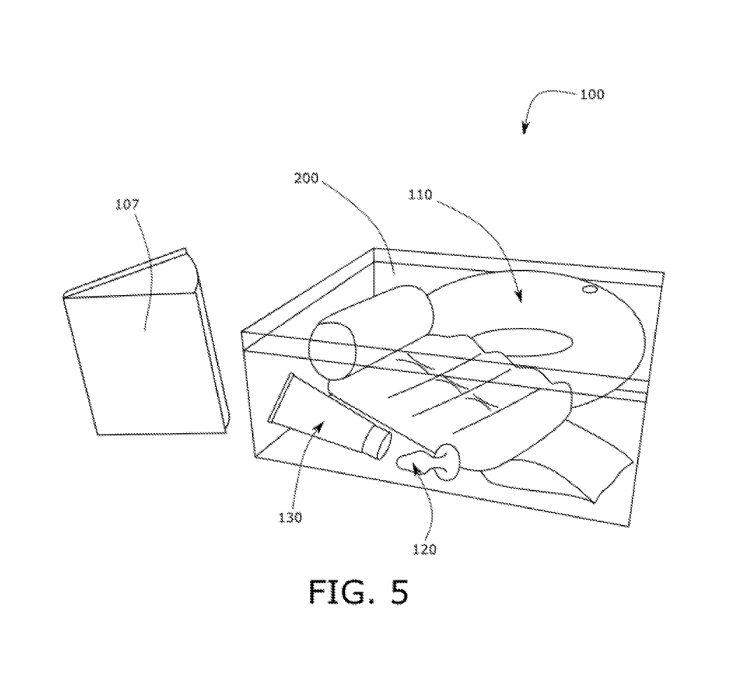

FIG. 5 shows a front perspective view of the kit 100 of FIG. 1, according to an embodiment of the present disclosure. As shown here, the kit 100 may further include a set of user instructions 107. The set of user instructions 107 may include educational information on rectal, anal, colon discomforts, infections or diseases to educate the user as to their situation, how to best handle them, and how to use each of the devices provided in the kit 100. Other variants of user instructions are also contemplated, such as manual guides and books. The books may include anecdotes, stories, instructional images, instructional diagrams, etc. Further, as shown, the kit 100 may further include a box 200 for containing the at least one cushion-device 110, the at least one cold-therapy device 120 and one or more medicaments 130 therein, or any variant thereof. In some embodiments, the box may further contain the at least one warm-therapy device 140 (FIG. 2) and the set of user instructions 105.

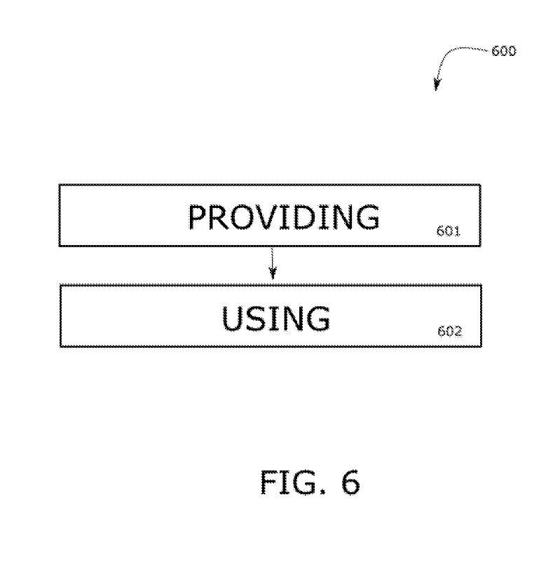

FIG. 6 is a flow diagram illustrating a method of using a kit for providing pain relief to a user suffering from hemorrhoids 600, according to an embodiment of the present disclosure. As illustrated, the method of using a kit for providing pain relief to a user suffering from hemorrhoids 600 may include the steps of: step one 601, providing the kit 100 as above; and step two 602, using one of the at least one cushion-device 110, the at least one cold-therapy device 120, and the at least one medicament 130 to provide pain relief from the hemorrhoids.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for kit 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A kit for providing pain relief to a user suffering from rectal, anal, colon discomforts, infections or diseases, the kit comprising:
at least one cushion-device, the at least one cushion-device being configured to provide comfort to at least one body-part of the user;
at least one cold-therapy device, the at least one cold-therapy device being configured to soothe inflammation associated with the rectal, anal, colon discomforts, infections or diseases; and
one or more medicaments, the medicaments configured for application to the at least one body-part of the user.

2. The kit of claim 1, further comprising a set of user instructions.

3. The kit of claim 2, wherein the set of user instructions includes educational information on rectal, anal, colon discomforts, infections or diseases.

4. The kit of claim 1, wherein the cushion-device is inflatable.

5. The kit of claim 4, wherein the cushion-device includes a donut-shape.

6. The kit of claim 5, wherein the cushion-device includes a front-surface and a rear-surface opposite the front-surface, and wherein the rear-surface includes an attachment-means configured to attach the cushion-device to a third surface.

7. The kit of claim 6, wherein the attachment-means is at least one suction-cup.

8. The kit of claim 7, wherein the third surface is at least one of a wall of a bathtub and a bottom of a bathtub.

9. The kit of claim 1, wherein the at least one cold-therapy device includes a rectal plug.

10. The kit of claim 9, wherein the rectal plug is made from an insulative-material.

11. The kit of claim 9, wherein the at least one cold-therapy device further includes underwear that has at least one pocket.

12. The kit of claim 11, further includes an ice-pack configured for removable-insertion into the at least one pocket.

13. The kit of claim 1, wherein a medicament of the one or more medicaments includes at least one lotion.

14. The kit of claim 13, wherein the at least one lotion includes one of the group consisting of hemp oil, aloe vera, and anti-inflammatory medicine.

15. The kit of claim 1, wherein a medicament of the one or more medicaments includes at least one suppository.

16. The kit of claim 15, wherein the at least one suppository includes phenylephrine.

17. The kit of claim 1, further comprising a box for containing the at least one cushion-device, the at least one cold-therapy device, and the one or more medicaments therein.

18. The kit of claim 1, further comprising at least one warm-therapy device.

19. A kit for providing pain relief to a user suffering from hemorrhoids, the kit comprising:
at least one cushion-device, the at least one cushion-device being configured to provide comfort to at least one body-part of the user;
at least one cold-therapy device, the at least one cold-therapy device being configured to soothe inflammation associated with the hemorrhoids;
one or more medicaments, the medicaments configured for application to the hemorrhoids;
a set of user instructions; and
a box for containing the at least one cushion-device, the at least one cold-therapy device, the at least one medicament, and the set of user instructions therein; and
wherein the set of user instructions includes educational information on hemorrhoids;
wherein the cushion-device is inflatable and includes a front-surface and a rear-surface opposite the front-surface, and the rear-surface includes;
at least one suction-cup configured to attach the cushion device to at least one of a wall of a bathtub and a bottom of a bathtub;
wherein the at least one cold-therapy device includes a rectal plug made from an insulative-material;
wherein the at least one cold-therapy device further includes underwear with at least one pocket;
wherein the at least one cold-therapy device further includes an ice-pack configured for removable-insertion into the at least one pocket;
wherein the at least one medicament includes at least one lotion including hemp oil; aloe vera; and an anti-inflammatory medicine; and
wherein the at least one medicament further includes at least one suppository.

20. A method of using a kit for providing pain relief to a user suffering from rectal, anal, colon discomforts, infections or diseases, the method comprising the steps of:
providing the kit for providing pain relief to the user suffering from the rectal, anal, colon discomforts, infections or diseases, the kit including:
at least one cushion-device, the at least one cushion-device being configured to provide comfort to at least one body-part of the user;
at least one cold-therapy device, the at least one cold-therapy device being configured to soothe inflammation associated with the rectal, anal, colon discomforts, infections or diseases; and
one or more medicaments, the medicaments configured for application to the at least one body-part of the user;
using one of the at least one cushion-device, the at least one cold-therapy device, and the at least one medicament to provide pain relief from the rectal, anal, colon discomforts, infections or diseases.
